(19) United States
(12) Patent Application Publication
(10) Pub. No.: US 2025/0366727 A1
(43) Pub. Date: Dec. 4, 2025

(54) MULTI-SENSOR OPTICAL BIOSENSOR DEVICE AND SYSTEM

(71) Applicant: KEHAAI, INC., Fremont, CA (US)

(72) Inventor: Vahram MOURADIAN, Fremont, CA (US)

(21) Appl. No.: 18/978,720

(22) Filed: Dec. 12, 2024

Related U.S. Application Data
(60) Provisional application No. 63/652,907, filed on May 29, 2024.

(51) Int. Cl. A61B 5/0225; A61B 5/00

(52) U.S. Cl. CPC A61B 5/02255; A61B 5/6826

(57) ABSTRACT

A multi-sensor optical biosensor system to determine one or more health parameters of a user, such as the user's blood pressure. The system includes a multi-sensor wearable device communicatively connected to a user compute device, which may be in communication with a remote compute device. The multi-sensor wearable device could include optical devices to generate a photoplethysmogram (“PPG”) signal that is transmitted to the user compute device. In some cases, the multi-sensor wearable device may be configured to select a PPG channel to transmit to the user's compute device from a plurality of PPG channels based on one or more selection parameters.

### RELATED APPLICATIONS

This application claims the benefit of U.S. Provisional Application Ser. No. 63/652,907 filed May 29, 2024 for a “Multi-Sensor Optical Biosensor Device and System,” which is hereby incorporated by reference in its entirety.

### BACKGROUND

Wearable devices allow users to monitor their health, and in some cases, allow remote health monitoring. Wearables come in many forms, such as activity trackers, smart rings, and smart watches. With wearables, users can collect personal health data that can lead to actionable insights, which can help the users to lead a healthier lifestyle. However, due at least in part to size limitations of wearables, there are many technical challenges that arise in design and implementation that need to be addressed.

### SUMMARY

According to one aspect, this disclosure relates to a wearable device that includes a plurality of photoplethysmogram (“PPG”) channels. In some cases, each of the plurality of PPG channels comprise at least one light emitter and at least one light receiver sensor, such as a photo diode (“PD”). The plurality of the PPG channels may include a single type (transmissive or reflective PPG biosensors), or both types of PPG biosensors intermixed (transmissive and reflective PPG biosensors). There is a non-transitory computer-readable memory having computer instructions stored thereon. The wearable device includes a processor in communication with the plurality of PPG channels and the computer-readable memory, wherein the computer instructions, when executed by the processor, causes the processor to perform operations comprising: generating respective PPG signals of the plurality of PPG channels; establishing one or more selection parameters to choose a selected PPG channel; choosing the selected PPG channel from the plurality of PPG channels based on which of the respective PPG signals best fits the one or more selection parameters; and transmitting data associated with the PPG signal of the selected PPG channel.

According to another aspect, this disclosure relates to a photoplethysmogram (“PPG”) ring configured to be worn on a finger of a user. In some cases, the PPG ring comprises a ring body including an inner body and an outer body. The ring may include a flexible circuit disposed between the inner body and the outer body of the ring body. For example, the flexible circuit may include a plurality of light emitters to selectively transmit light and a plurality of light receiver sensors arranged along the inner surface to detect the transmitted light, wherein the plurality of light emitters and a plurality of light receiver sensors define a plurality of PPG channels. The PPG ring includes a non-transitory computer-readable memory having computer instructions stored thereon. There may be a processor in communication with the plurality of PPG channels and the computer-readable memory, wherein the computer instructions, when executed by the processor, causes the processor to perform operations comprising: generating respective PPG signals of the plurality of PPG channels; establishing one or more selection parameters to choose a selected PPG channel; choosing a selected PPG channel from the plurality of PPG channels based on which of the respective PPG signals best fits the one or more selection parameters; and transmitting data associated with the PPG signal of the selected PPG channel.

According to a further aspect, this disclosure relates to a method of selecting a photoplethysmogram (“PPG”) channel. The method includes the step of receiving respective PPG signals of a plurality of PPG channels such that the PPG signals were generated based on one or more pair(s) of light emitters LED (Light-Emitting Diode) and light receiver sensors (PD). In this embodiment, the method may include establishing one or more selection parameters to choose a selected PPG channel and choosing the selected PPG channel from the plurality of PPG channels based on which of the respective PPG signals best fits the one or more selection parameters. The data associated with the PPG signal of the selected PPG channel may then be transmitted.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements. The detailed description particularly refers to the accompanying figures in which:

FIG. 1 is a simplified block diagram of at least one embodiment of a multi-sensor optical biosensor system;

FIG. 2 is a simplified block diagram of at least one embodiment of a multi-sensor wearable device of the system of FIG. 1;

FIG. 3 is a simplified block diagram of at least one embodiment of a user and/or analysis compute device of the system of FIG. 1;

FIG. 4 is a perspective view of at least one embodiment of the multi-sensor wearable device of the system of FIG. 1;

FIG. 4A is a perspective view of at least one embodiment of the multi-sensor wearable device showing a plurality of light emitters and/or light receiver sensors of the system of FIG. 1;

FIG. 5 is a side view of the multi-sensor wearable device of FIG. 4;

FIG. 6 is an exploded view of the multi-sensor wearable device of FIG. 4;

FIG. 7 is a side cross-sectional view of the multi-sensor wearable device of FIG. 4;

FIG. 8 a table showing example channels defined by the multi-sensor wearable device of FIG. 4;

FIG. 9 is a simplified block diagram of at least one embodiment of a method for determining a signal channel to transmit that may be executed by the multi-sensor wearable device of FIG. 1;

FIG. 10 is a simplified block diagram of at least one embodiment of a method for determining blood pressure that may be executed by the wearable device and/or user compute device of FIG. 1;

FIG. 11 is a simplified block diagram of at least one embodiment of a method for analyzing signal channel data to determine blood pressure that may be executed by the analysis compute device of FIG. 1;

FIG. 12 is a simplified block diagram of at least one embodiment of a method for determining blood pressure that may be executed by the analysis compute device of FIG. 1;

FIG. 13 illustrates examples of analysis regarding PPG signals that may be performed by the analysis compute device of FIG. 1;

FIG. 14 illustrates an example table defining an embodiment of user descriptive points (UDP) that could be used by the analysis compute device as part of determining the blood pressure of FIG. 1; and

FIGS. 15-21 are diagrams illustrating at least one embodiment of a band-pass filter that could be used to filter channel signals of the system of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

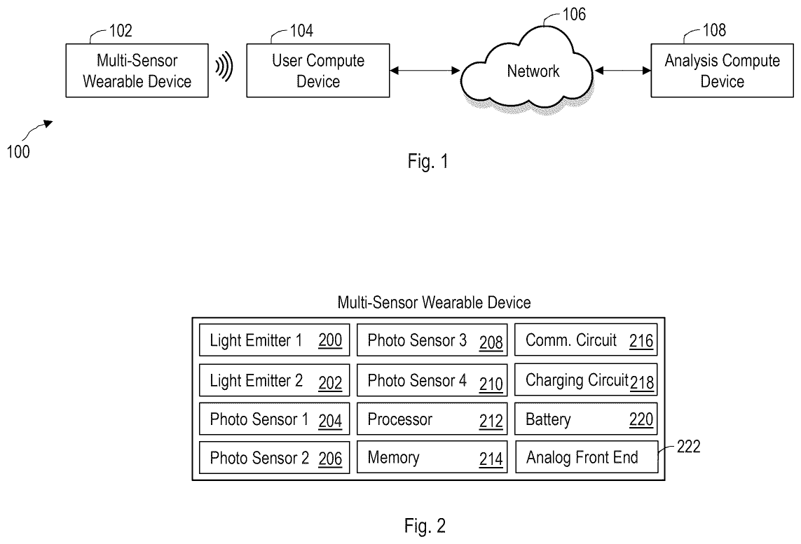

Referring now to FIG. 1, a multi-sensor optical biosensor system 100 to determine one or more health parameters of a user, such as, for example, the user's blood pressure. In the illustrative embodiment, the system 100 includes a multi-sensor wearable device 102 communicatively connected to a user compute device 104. The multi-sensor wearable device 102 is an electronic device that is wearable on or attached to the user's body. For example, in some cases, the multi-sensor wearable device 102 could be embodied as a ring. In some embodiments, the multi-sensor wearable device 102 is configured to take optical health measurements of the user. For example, the multi-sensor wearable device 102 could include optical devices to generate a photoplethysmogram (PPG) signal that is transmitted to the user compute device 104. In some embodiments, the multi-sensor wearable device 102 could be configured to wirelessly communicate the PPG signal with the user compute device 104. By way of example only, the multi-sensor wearable device could be configured to communicate with the user compute device 104 within a wireless range, such as using Bluetooth™ low energy.

The user compute device 104 is configured to receive, among other things, the PPG signal from the multi-sensor wearable device 102. For example, the user compute device 104 may be a mobile device, such as the user's phone or tablet. As shown, the user compute device 104 is communicatively connected by a network 106 to an analysis compute device 108. In some embodiments, the user compute device 104 is configured to send at least a portion of the optical health measurements, such as the PPG signal, received from the multi-sensor wearable device 102 to the analysis compute device 108. The user compute device 104 may receive an analysis of the PPG signal from the analysis compute device 108. For example, the user compute device 104 may receive a blood pressure measurement from the analysis compute device 108. Depending on the circumstances, an analysis of the PPG signal could be done on-board the user compute device 104, and the analysis compute device 108 may be optional.

In some embodiments, in operation, the multi-sensor optical biosensor system 100 may be configured to take optical measurements of the user with the multi-sensor wearable device 102 to generate a PPG signal. The user compute device 104 wirelessly receives the PPG signal, and sends at least a portion of the PPG signal to the analysis compute device 108 via the network 106 to determine a blood pressure. The analysis compute device 108 sends the blood pressure measurement to the user compute device 104, and this may be displayed on a screen of the device 104.

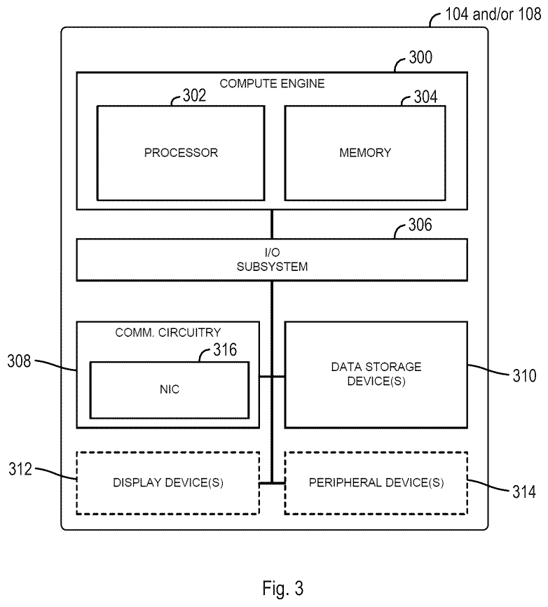

Referring now to FIG. 2, there is shown an embodiment of the multi-sensor wearable device 102. In the illustrative embodiment, the multi-sensor wearable device 102 includes a plurality of light emitters, such as light emitter 1 200, and light emitter 2 202, a plurality of light receiver sensors, such as light receiver sensor 1 204, light receiver sensor 2 206, light receiver sensor 3 208, light receiver sensor 4 210, a processor 212, a memory 214, a communication circuit 216, a charging circuit 218, a battery 220, and analog front end (AFE) 222. In some embodiments, the light emitters 200, 202 could be embodied as LEDs, such as green LEDs, red LEDs, infrared (“IR”) LEDs. For example, the light emitters 200, 202 may be configured to emit light towards human tissue, which is may be transmitted through the tissue and/or back-scattered or reflected from the tissue. In embodiments in which the multi-sensor wearable device 102 is a ring, the light emitters 200, 202 could be configured to transmit light through the user's finger and/or reflect light off the user's finger. The light receiver sensors 204, 206, 208, 210 could be arranged with respect to the light emitters 200, 202 to sense light that is transmitted through the user's tissue and light that is reflected off the tissue. By way of example only, if the one or more of the light emitters 200, 202 were green LEDs and/or red LEDs that tend to reflect light off the user's tissue, one or more light receiver sensors 204, 206, 208, 210 could be arranged where the light will be reflected to sense the reflected light. In another example, if one or more of the light emitters 200, 202 were IR LEDs, which tend to transmit through the user's tissue, one or more light receiver sensors 204, 206, 208, 210 could be arranged on an opposite side of the tissue to sense the light transmitted through the tissue. Although two light emitters 200, 202 are shown for purposes of example, more than two light emitters 200, 202 could be provided depending on the circumstances; likewise, the multi-sensor wearable device may include a single light emitter in some cases. Although four light receiver sensors 204, 206, 208, 210 are shown for purposes of example, less than or more than four light receiver sensors could be included in the multi-sensor wearable device depending on the circumstances.

As shown, the multi-sensor wearable device includes a processor 212 and memory 214 to perform one or more of the functions described herein. Although the processor 212 and memory 214 are shown separately for purposes of example, they may be embodied as a single device such as a system-on-a-chip (SOC), or other integrated system or device. In embodiments, the processor 212 is capable of receiving, e.g., from the memory 214, a set of instructions, such as firmware code, which when executed by the processor 212 cause the multi-sensor wearable device 102 to perform one or more operations described herein.

The processor 212 may be embodied as any type of processor such as a single or multi-core processor(s), a microcontroller, or other processor or processing/controlling circuit. In some embodiments, the processor 212 may be embodied as, include, or be coupled to an FPGA, an application specific integrated circuit (ASIC), reconfigurable hardware or hardware circuitry, or other specialized hardware to facilitate performance of the functions described herein. In embodiments, the processor 212 is further capable of receiving, one or more signals from external sources, e.g., from the light receiver sensors 204, 206, 208, 210 and/or communications circuit 216. As one will appreciate, a signal may contain encoded instructions and/or information. In some embodiments, once received, such a signal may first be stored, e.g., in the memory 214 before the processor 212 operates on a received signal. Likewise, the processor 212 may generate one or more output signals, which may be transmitted to an external device, e.g., the user compute device 104 via the communication circuitry 216. One will appreciate that the form of a particular signal will be determined by the particular encoding a signal is subject to at any point in its transmission (e.g., a signal stored will have a different encoding that a signal in transit, or, e.g., an analog signal will differ in form from a digital version of the signal prior to an analog-to-digital (A/D) conversion).

The memory 214 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the memory 214 may be integrated into the processor 212. In operation, the memory 214 may store various firmware and data used during operation such as PPG signal data, applications, libraries, and drivers.

The communication circuit 216 may be embodied as any communication circuit, device, or collection thereof, capable of enabling wireless communications from the multi-sensor wearable device 102 to an external device, such as the user compute device 104. The communication circuit 216 may be configured to use any one or more wireless technologies and associated protocols (e.g., Bluetooth®, Wi-Fi®, WiMAX, etc.) to effect such communication. In some embodiments, the communication circuit 216 may be embodied as part of a system-on-a-chip (SoC) that includes one or more processors, or included on a multichip package that also contains one or more processors.

The charging circuit 218 is configured to recharge the battery 220, which supplies electrical power to the other components of the multi-sensor wearable device 102. The charging circuit 218 may be connected with an external power source with a wired or wireless connection to recharge the circuit.

The analog front end (AFE) 222 is configured to perform signal acquisition and/or conditioning, such as acquisition and/or conditioning of PPG signal data. By way of example only, the AFE 222 could be embodied as the AFE4950 by Texas Instruments of Dallas, Texas.

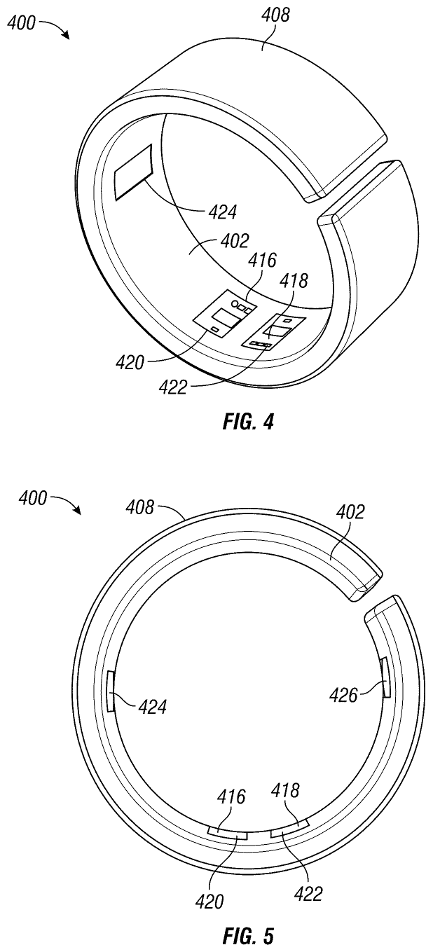

Referring now to FIG. 3, there is shown an example of the user compute device 104 and/or analysis compute device 108. As shown, the user compute device 104 and/or analysis compute device 108 includes a compute engine 300. There is an input/output (I/O) subsystem 306, communication circuitry 308, and one or more data storage devices 310. In some embodiments, the user compute device 104 and/or analysis compute device 108 may include one or more display devices 312 and/or one or more peripheral devices 314 (e.g., a mouse, a physical keyboard, etc.). In some embodiments, one or more of the illustrative components may be incorporated in, or otherwise form a portion of, another component. The compute engine 300 may be embodied as any type of device or collection of devices capable of performing various compute functions described below. In some embodiments, the compute engine 300 may be embodied as a single device such as an integrated circuit, an embedded system, a field-programmable gate array (FPGA), a system-on-a-chip (SOC), or other integrated system or device. Additionally, in the illustrative embodiment, the compute engine 300 includes or is embodied as a processor 302 and a memory 304. The processor 302 may be embodied as any type of processor capable of performing the functions described herein. For example, the processor 302 may be embodied as a single or multi-core processor(s), a microcontroller, or other processor or processing/controlling circuit. In some embodiments, the processor 302 may be embodied as, include, or be coupled to an FPGA, an application specific integrated circuit (ASIC), reconfigurable hardware or hardware circuitry, or other specialized hardware to facilitate performance of the functions described herein. The memory 304 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the memory 304 may be integrated into the processor 302. In operation, the memory 304 may store various software and data used during operation such as PPG signal and/or blood pressure data, applications, libraries, and drivers.

The compute engine 300 is communicatively coupled to other components of the user compute device 104 and/or analysis compute device 108 via the I/O subsystem 306, which may be embodied as circuitry and/or components to facilitate input/output operations with the compute engine 300 (e.g., with the processor 302 and the main memory 304). For example, the I/O subsystem 306 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 306 may form a portion of a system-on-a-chip (SoC) and be incorporated, along with one or more of the processor 302, the memory 304 into the compute engine 210.

The communication circuitry 308 may be embodied as any communication circuit, device, or collection thereof, capable of enabling communications over the network 106 between the user compute device 104 and/or analysis compute device 108 and/or another device. The communication circuitry 308 may be configured to use any one or more communication technology (e.g., wired or wireless communications) and associated protocols (e.g., Ethernet, Wi-Fi®, WiMAX, Bluetooth®, etc.) to effect such communication. The illustrative communication circuitry 308 includes a network interface controller (NIC) 316. The NIC 316 may be embodied as one or more add-in-boards, daughter cards, network interface cards, controller chips, chipsets, or other devices that may be used by the user compute device 104 and/or analysis compute device 108 to connect with each other and/or another compute device. In some embodiments, the NIC 316 may be embodied as part of a system-on-a-chip (SoC) that includes one or more processors, or included on a multichip package that also contains one or more processors. In some embodiments, the NIC 316 may include a local processor (not shown) and/or a local memory (not shown) that are both local to the NIC 316. Additionally or alternatively, in such embodiments, the local memory of the NIC 316 may be integrated into one or more components of the user compute device 104 and/or analysis compute device 108 at the board level, socket level, chip level, and/or other levels. In the illustrative embodiment, the user compute device 104 and analysis compute device 108 are in communication via the network 106, which may be embodied as any type of wired or wireless communication network, including global networks (e.g., the internet), wide area networks (WANs), local area networks (LANs), digital subscriber line (DSL) networks, cable networks (e.g., coaxial networks, fiber networks, etc.), cellular networks (e.g., Global System for Mobile Communications (GSM), Long Term Evolution (LTE), Worldwide Interoperability for Microwave Access (WiMAX), 3G, 4G, 5G, etc.), a radio area network (RAN), or any combination thereof.

Each data storage device 310, may be embodied as any type of device configured for short-term or long-term storage of data such as, for example, memory devices and circuits, memory cards, hard disk drives, solid-state drives, or other data storage device. Each data storage device 310 may include a system partition that stores data and firmware code for the data storage device 310 and one or more operating system partitions that store data files and executables for operating systems.

Each display device 312 may be embodied as any device or circuitry (e.g., a liquid crystal display (LCD), a light emitting diode (LED) display, a cathode ray tube (CRT) display, etc.) configured to display visual information (e.g., text, graphics, etc.) to a user. In some embodiments, a display device 312 may be embodied as a touch screen (e.g., a screen incorporating resistive touchscreen sensors, capacitive touchscreen sensors, surface acoustic wave (SAW) touchscreen sensors, infrared touchscreen sensors, optical imaging touchscreen sensors, acoustic touchscreen sensors, and/or other type of touchscreen sensors) to detect selections of on-screen user interface elements or gestures from a user.

In the illustrative embodiment, the components of the user compute device 104 and analysis compute device 108 are housed in a single unit. However, in other embodiments, the components may be in separate housings. For example, the analysis compute device 108 may be in separate racks of a data center, and/or spread across multiple data centers or other facilities.

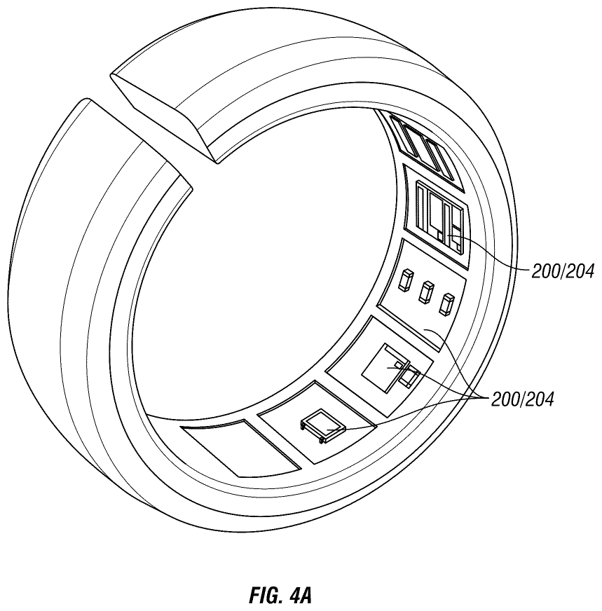

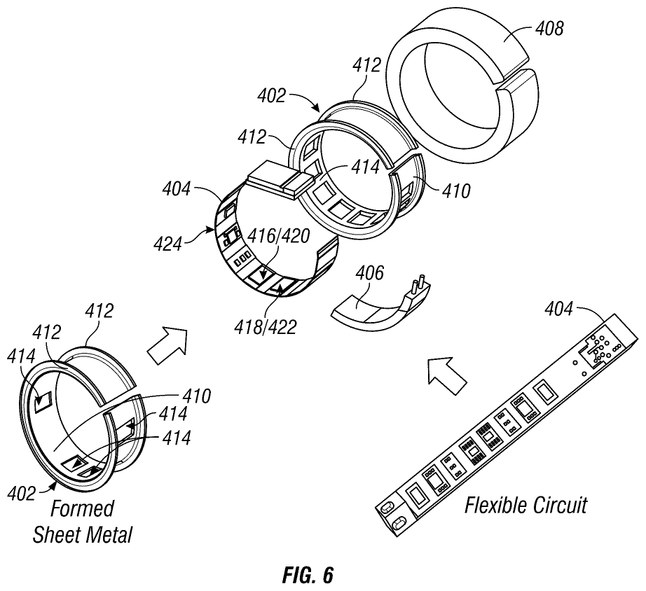

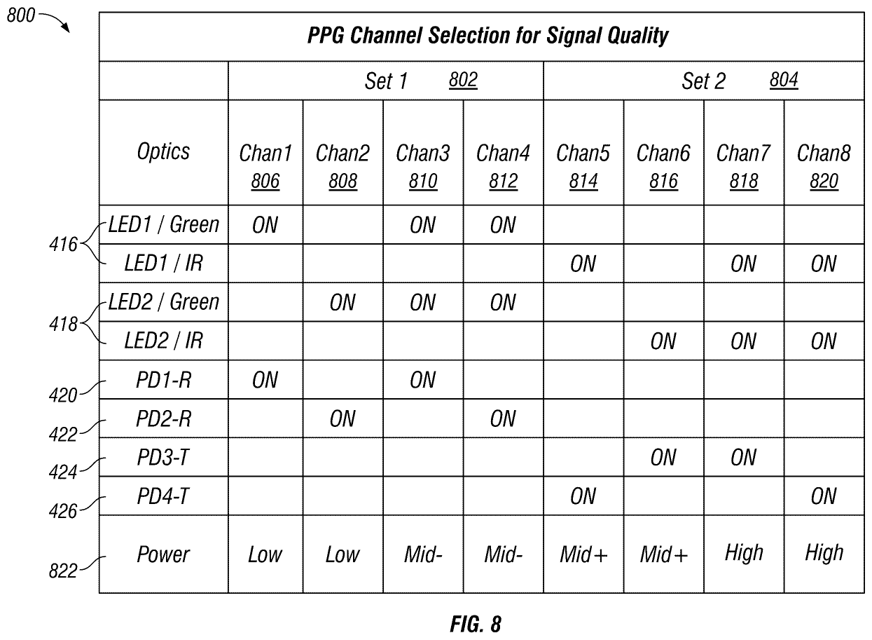

Referring now to FIGS. 4-7, there is shown an example of the multi-sensor wearable device 102 embodied as a ring 400 that could be worn on one of the user's fingers. In the embodiment shown, the ring 400 includes an inner body 402, a flexible circuit 404 and curved battery 406 that surround the inner body 402, and an outer body 408 that surrounds the flexible circuit 404 and curved battery 408. As shown, the ring 400 is shaped substantially as a circle, but could be a polygon or other shape that could be worn on a user's finger. This disclosure is not intended to be limited to the specific shape shown in FIGS. 4-7, but the ring 400 could be embodied in a wide variety of different shapes and designs that are all encompassed within this disclosure.

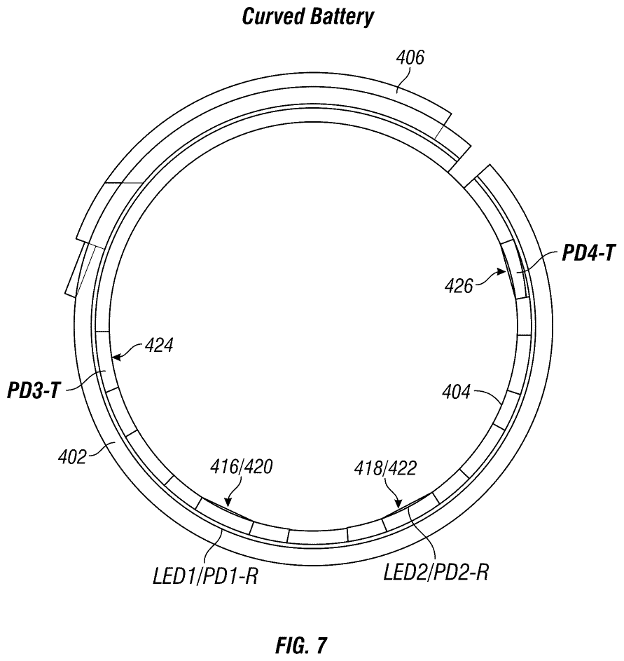

In the embodiment shown, the inner body 402 has a central annular portion 410 and a peripheral annual flanges 412 (see FIG. 6). In some embodiments, the inner body 402 could be formed from sheet metal or other sturdy material-made piece, such as plastic, could be used depending on the circumstances. The outer body 408 could be formed from a variety of metal and polymeric materials, such as an over molded plastic or natural/synthetic rubber. In some embodiments, the flexible circuit 204 could be formed from a flexible PCB material that allows the flexible circuit 404 to bend around the inner body 402. In some embodiments, the curved battery 406 is curved corresponding with the curvature of the inner body 402.

The width of the central annular portion 410 is dimensioned to receive the width of the flexible circuit 404. The depth of the peripheral annular flanges 412 are dimensioned to receive the thickness of the flexible circuit 404 and curved battery 406 so that the flexible circuit 204 and curved battery 406 does not extend above the peripheral annular flanges. In this manner, the flexible circuit 204 and curved battery 406 are housed between the inner body 402 and the outer body 408.

As shown, there are a plurality of openings 414 in the inner body 402 through which light emitters or LEDs 200, 202 and/or light receiver sensors 204, 206, 208, 210 can emit or sense light, respectively. In the embodiment shown, the flexible circuit 404 includes a first LED 416 and a second LED 418. Depending on the circumstances, the first LED 416 and second LED 418 may selectively emit green, red and/or IR light. As shown, the first LED 416 and second LED 418 are adjacent each other. In the embodiment shown, the flexible circuit 404 also includes a first light receiver sensor 420, a second light receiver sensor 422, a third light receiver sensor 424, and a fourth light receiver sensor 426. As shown, the first light receiver sensor 420 is integrated with the first LED 416 and the second light receiver sensor 422 is integrated with the second LED 418. However, depending on the circumstances, these could be separate components. Although two LEDs and four light receiver sensors are shown for purposes of example, more of less LEDs and/or light receiver sensors could be provided depending on the circumstances. For example, in FIG. 4A, there is shown an example embodiment with additional light receiver sensors 204, 206, 208, 210 and/or LEDs 200, 202 compared to the example shown in FIG. 4.

In some embodiments, the first light receiver sensor 420 and the second light receiver sensor 422 are configured to detect light reflected from the user's finger. The third light receiver sensor 424 and fourth light receiver sensor 426 may be configured to detect light transmitted through the user's finger. In with configuration, the first light receiver sensor 420 and the second light receiver sensor 422 are spatially arranged on the ring to a position where the light would be reflected from the first and second LEDs 416, 418. As shown, the third light receiver sensor 424 and fourth light receiver sensor 426 are spatially arranged on approximately opposing sides of the ring 400. Typically, the third light receiver sensor 424 and fourth light receiver sensor 426 are positioned where light (e.g., IR light) would most likely be transmitted through the user's finger from the first LED 416 and/or the second LED 418.

The processor 212 is configured to selectively turn on/off the LEDs 416, 418 and light receiver sensors 420, 422, 424, 426 to generate, among other things, a PPG signal. However, there is a technical problem presented regarding which of the LEDs 416, 418 and light receiver sensors 420, 422, 424, 426 should be turned/off when the user rotates the ring 400. The rotation of the ring 400 adjusts the relative position of the LEDs 416, 418 and light receiver sensors 420, 422, 424, 426 with respect to the user's blood vessels/arteries from which the measurements are taken. There is also a tradeoff between how many of the LEDs 416, 418 and light receiver sensors 420, 422, 424, 426 should be turned on and the energy usage (i.e., battery life). Additionally, there may be different energy consumptions for detecting light reflected from the user's finger (e.g., green/red light) versus light transmitted through the user's finger (e.g., IR light).

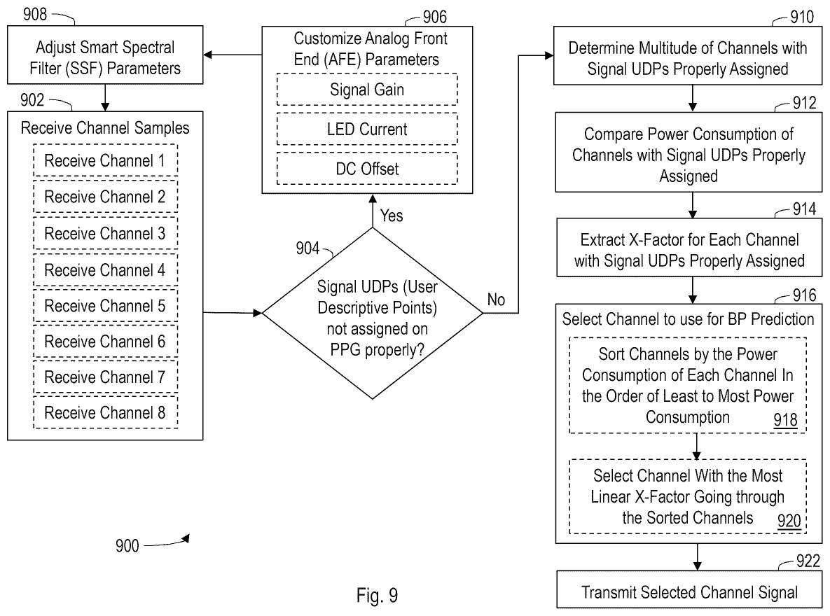

Referring now to FIG. 8, there is a table 800 showing an example ring 400 configuration in which the LEDs 416, 418 and light receiver sensors 420, 422, 424, 426 are defined as a first set 802 related to measurements made from reflected light and a second set 804 related to measurements made from light transmitted through the user's finger. As shown, the first set 802 is defined as the first LED 416 and second LED 418 transmitting visible light (e.g., green or red) and the first light receiver sensor 420 and the second light receiver sensor 422 configured to detect reflected light. The second set 804 is defined as the first LED 416 and second LED 418 transmitting non-visible light (e.g., IR light) and the third light receiver sensor 424 and the fourth light receiver sensor 424 configured to detect light transmitted through the user's finger.

Within the first set 802, the table 800 defines a first channel 806 with the first LED 416 transmitting visible light (e.g., green or red light) and the first light receiver sensor 420 detecting reflected light, a second channel 808 with the second LED 418 transmitting visible light (e.g., green or red light) and the second light receiver sensor 422 detecting reflected light, a third channel 810 with the first and second LEDs 416, 418 transmitting visible light (e.g., green or red light) and the first light receiver sensor 420 detecting reflected light, and a fourth channel 812 with the first and second LEDs 416, 418 transmitting visible light (e.g., green or red light) and the second light receiver sensor 422 detecting reflected light.

Within the second set 804, the table 800 defines a fifth channel 814 with the first LED 416 transmitting non-visible light (e.g., IR light) and the third light receiver sensor 424 detecting transmitted light, a sixth channel 816 with the second LED 416 transmitting non-visible light (e.g., IR light) and the fourth light receiver sensor 426 detecting transmitted light, a seventh channel 818 with the first and second LEDs 416, 418 transmitting non-visible light (e.g., IR light) and the third light receiver sensor 424 detecting transmitted light, and an eighth channel 820 with the first and second LEDs 416, 418 transmitting non-visible light (e.g., IR light) and the fourth light receiver sensor 426 detecting transmitted light. Although the table 800 shows an example with eight (8) channels, there could be more or less channels depending on the circumstances.

The table 800 provides example energy consumption ratings 822 for each of the channels 806, 808, 810, 812, 814, 816, 818, 820. In the example shown, the first and second channels 806, 808 are rated as low energy consumption. As shown, the third and fourth channels 810, 812, are rated as mid-energy consumption, which is higher than the first and second channels 806, 808, but less than the other channels 814, 816, 818, 820. The table 800 indicates that the fifth and sixth channels 814, 816 are rated as mid+ energy consumption, which is more than the channels 806, 808, 810, 812 in the first set 802 and less than the other channels 818, 820. As shown, the seventh and eighth channels 818, 820 are rated as high energy consumption, which is the highest energy of all channels.

In some embodiments, the processor 212 is configured to determine which of the LEDs 416, 418 and light receiver sensors 420, 422, 424, 426 to turn on/off to generate a high quality health-related measurement, such as PPG signal, at the lowest power consumption rating. For example, the processor 212 may select the first or second channel 806 or 808 if those channels generate a PPG signal of sufficient quality because those channels 806, 808 are the lowest energy consumption. However, if the quality of the signal for the first and second channels 806, 808 is insufficient, the processor 212 may select a different channel, such as the third, fourth, fifth, sixth, seventh, eighth channels 812, 814, 816, 818, 820 even though those channels have a higher energy consumption.

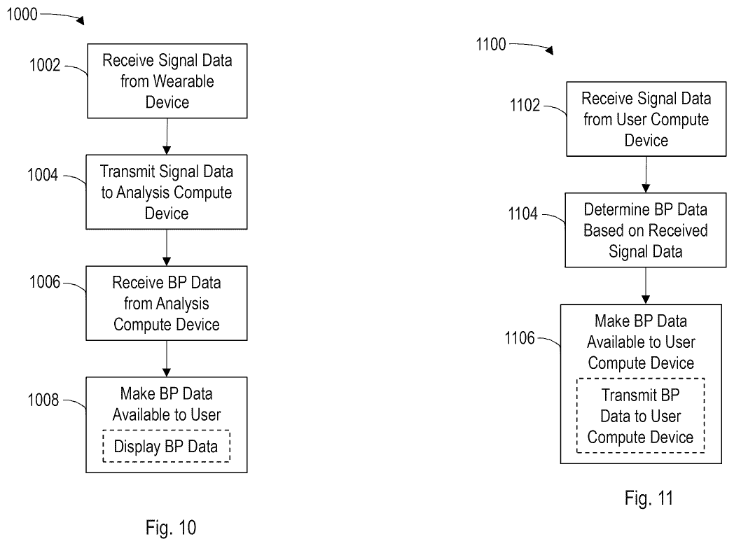

Referring now to FIG. 9, in some embodiments, the processor 212 may execute a method 900 to determine which channel to select to generate a health-related measurement, such as a PPG signal, for transmission to the user compute device 104. As shown, the method 900 begins with block 902 in which the processor turns on/off the channels 806, 808, 810, 812, 814, 816, 818, 820 to receive channel signal samples to evaluate which channel to select. Depending on the circumstances, the processor 212 could sequentially turn on/off channels 806, 808, 810, 812, 814, 816, 818, 820 in an order of increased energy consumption until a channel with sufficient signal quality is found, and that channel is selected. In some embodiments, one or more groups of channels 806, 808, 810, 812, 814, 816, 818, 820 and/or all channels 806, 808, 810, 812, 814, 816, 818, 820 are turned on/off to obtain signal samples to determine which channel has sufficient signal quality at the lowest energy consumption. In the embodiment shown, the method 900 advances to block 904 in which a determination is made whether any of the signal user descriptive points (UDPs) are not assigned on PPG properly. If there are signals where UDPs are not assigned properly, the processor 212 proceeds to block 906 in which one or more analog front end (AFE) parameters are customized, such as signal gain, LED current, and/or DC offset, one or more smart spectral filter (SSF) parameters may be adjusted (block 908), and advance back to block 902.

If at least one of the channels 806, 808, 810, 812, 814, 816, 818, 820 have a signal UDPs assigned on PPG properly, the method 900 proceeds to block 910 in which the processor 212 determines multitude of channels with signal UDPs properly assigned. The method advances to block 912 in which there is a comparison of power consumption between channels with signal UDPs that are properly assigned.

In some embodiments, the method 900 includes the step of extracting X-factor data 914 that can be used to determine the channel to select. In some cases, for example, the processor 212 may use machine learning, at least in part, to determine the channel to select. For example, some users may have a pattern of rotating the ring 400 to the same position, and the processor 212 may be configured to use machine learning to extract smart data to adapt which channel is selected based on usage patterns on which the user wears the ring 400. By way of example, the X-factor data could be a factor when evaluating which channel to select. The method 900 advances to block 916 to select which channel to transmit to the user compute device 104 to predict a health-related measurement, such as blood pressure. In the example shown, the channel selection based be based on sorting channels by power consumption of each channel in the order of least to most power consumption (block 918). The channel will then be selected that has the most linear X-factor going through the sorted channels. Once the channel is selected, the method 900 proceeds to block 920 in which the channel signal (e.g., PPG signal) is transmitted to the user compute device 104 (block 922). In some embodiments, the method 900 then periodically loops back to block 902 to sampling channels. Though the operations of the method 900 are described in a particular sequence, it should be understood that in other embodiments, operations may be performed in a different order and/or in parallel.

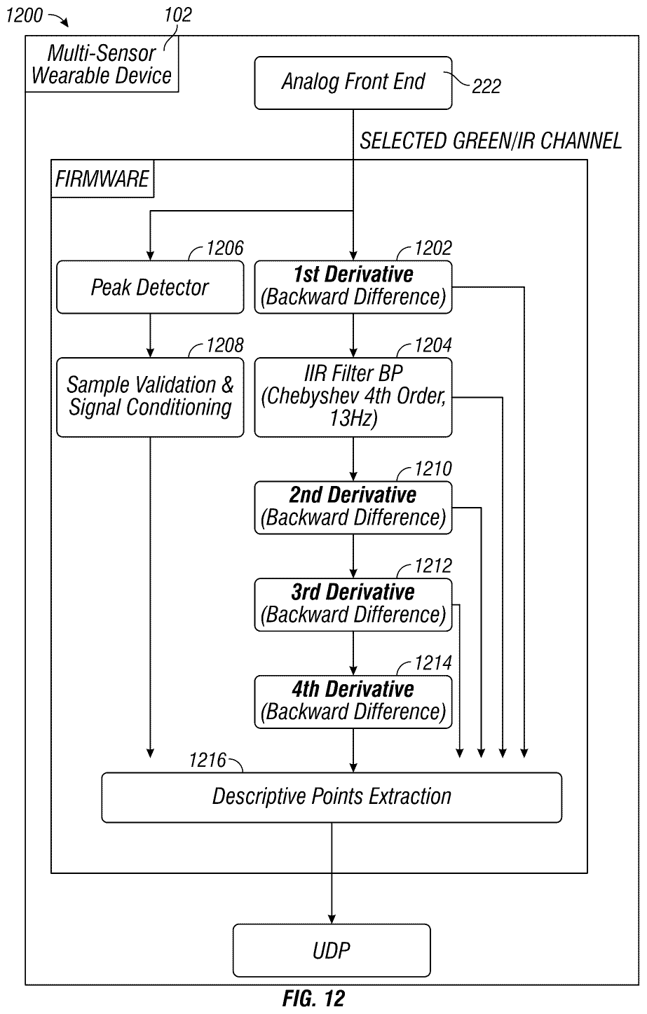

Referring now to FIG. 10, in some embodiments, the user compute device 104 may execute a method 1000 to make blood pressure data available to the user. In the embodiment shown, the method 1000 begins with block 1002 in which signal data (e.g., PPG signal) from the ring 400 is received by the user compute device 104. In some embodiments, the user compute device 104 receives the signal data wirelessly from the ring 400, such as through Bluetooth™ Low Energy (BLE). The method 1000 proceeds to block 1004 in which the signal data is transmitted to the analysis compute device 108, such as via the network 106. The method then advances to block 1006 in which blood pressure (BP) data is received from the analysis compute device 108. As discussed herein, in some embodiments, the determination of blood pressure (or other data) may be computed on the user compute device 104 instead of (or in addition to) the analysis compute device 108. The method 1000 proceeds to block 1008 in which the BP data is provided to the user, such as by displaying the BP data on a screen of the user compute device 104.

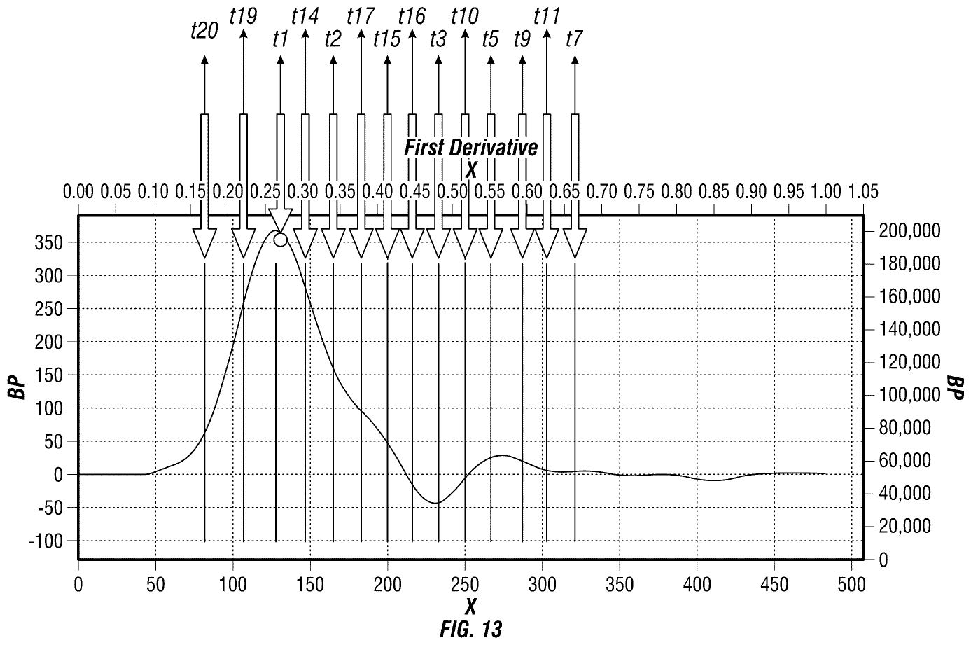

FIG. 11 shows an example method 1100 that may be executed by the analysis compute device 108. In the example shown, the method 1100 starts at block 1102 in which the analysis compute device 108 receives the signal data, such as PPG signal data, from the user compute device 104. The method 1100 proceeds to block 1104 in which BP data is determined based on the signal data. The analysis compute device 108 then makes the BP data available to the user compute device 108 (block 1106), such as by transmitting the BP data via the network 106.

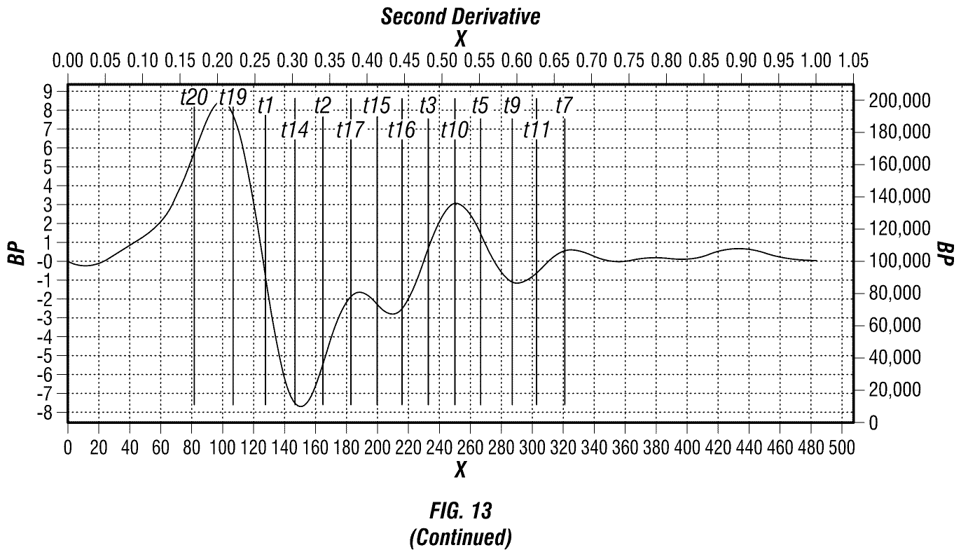

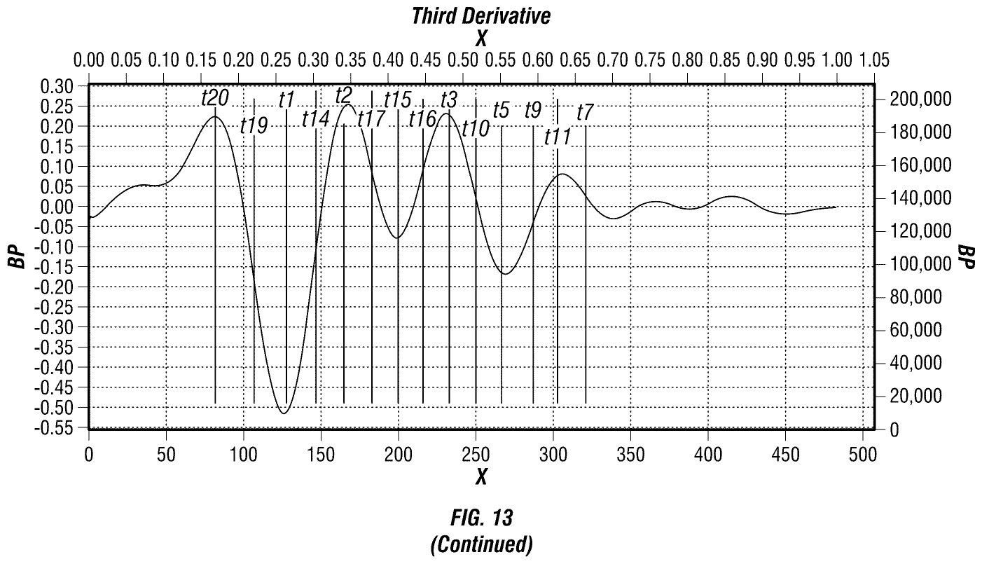

FIG. 12 illustrates a method 1200 that may be executed by the multi-sensor wearable device 102 (and/or the analysis compute device 108 and/or the user compute device 104) to determine one or more user descriptive points (UDP) with the analog front end (AFE) 222 from a PPG signal. In the embodiment shown, the method 1200 begins at block 1202 in which the 1 st derivative is calculated over the sampled points (e.g., 128/256/512) on the PPG signal. The example method 1200 proceeds to block 1204 in which there is infinite impulse response (IIR) filtering. In some embodiments, for example, the IIR filter could be cascaded Chebyshev 4th order filters with a cutoff frequency of 13 Hz. In the embodiment shown, the method 1200 advances to a 2^{nd} derivative 1210, which advances to the 3^{rd} derivative 1212, and then the 4^{th} derivative 1214. FIG. 13 illustrates examples of a PPG signal, 1^{st} derivative, 2^{nd} derivative, and 3^{rd} derivative signals. The method then advances to block 1216.

The method 1200 also advances to block 1206 in which the peak of the signal is detected. In some embodiments, for every 2 seconds interval of the green channel PPG signal average level is calculated, and the interval value of “delta” is calculated as (average−minimum)*0.75. Two phases (“looking for a maximum” and “looking for a minimum”) may be used for PPG wave detection. During the phase of “looking for a minimum” the minimum is found then the input signal is below Maximum Value (calculated in block 1206)−“delta” value. When the minimum is found, the phase changes to “looking for a maximum” and data about the signal position is stored. The minimum Value is set to the current signal value. During the phase of “looking for a maximum”, the maximum is found then the input signal is above Minimum Value (calculated in block 1206)+ “delta” value. Minimum and maximum signal values and positions are adjusted on every sample of input data if the input data is less or greater than the current signal value. If the time difference between the two last minimums is less than 2 seconds, a new PPG wave found event is sent out. Signal position timestamp and amplitude are stored. Maximum Value is set to current signal value. The phase changed to “looking for a minimum”.

The method 1200 then proceeds to block 1208 in which the multi-sensor wearable device 102 (and/or analysis compute device 108) performs sample validation/signal conditioning. For example, a heart rate pulse wave (further HR sample) is the PPG signal between two consecutive minimums. The HR sample may be considered invalid if: (1) the sample length is less than 0.25 sec; (2) the sample length is greater than 2 sec; (3) the signal peak is not located between two consecutive minimums; or (4) the signal amplitude (difference between last signal maximum and previous signal minimum) is less than defined by settings value signal slope (signal difference between current and previous minimums divided amplitude) is more than defined by settings value. The method 1200 then advances to block 1216 in which a user descriptive points (UDP) are determined.

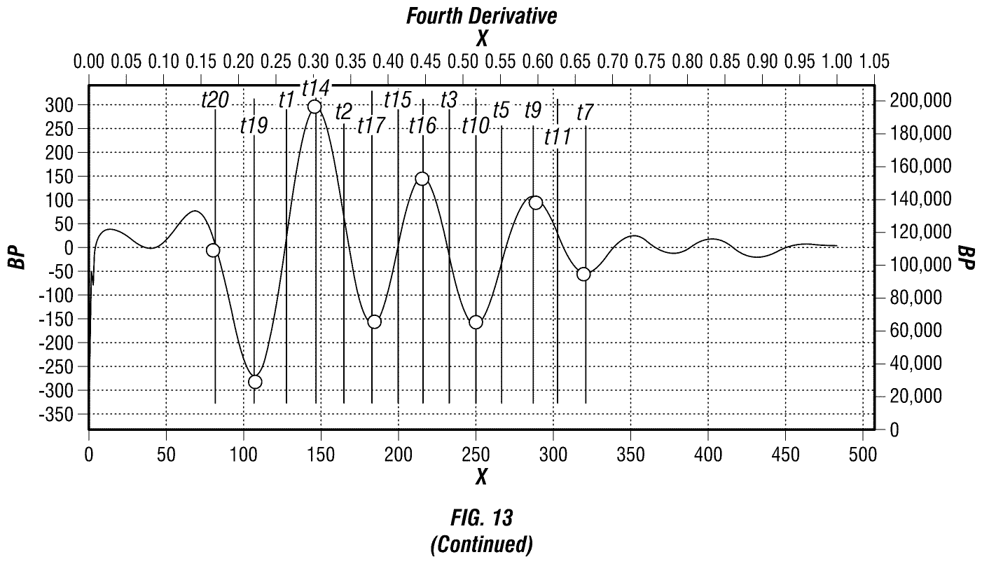

FIG. 14 describes a definition for UDP according to at least one embodiment. In some cases, for every point 1^{st} derivative at the point of input signal and timestamp are stored and can be used as parameters in equation on the next step. In some cases, there could be a custom model/equation applied to the blood pressure data. For example, the equation for calculation systolic and diastolic blood pressure has the form: BP=B+A*(v0/v1)*((v2−v3)/(v4−v5)). The values of A, B and links to points (v0, v1, v2, v3, v4, v5) transferred through the BLE from mobile app. One or more values could be discarded. For example, the first N (externally tunable) values may be discarded. In some embodiments, values that do not match the specified range are discarded. By way of example only, systolic values outside 50-250 could be discarded; likewise, diastolic values outside 20-200 may be discarded. Additionally, in some cases, if the difference between is outside between systolic and diastolic values are outside 10-120, the value may be discarded.

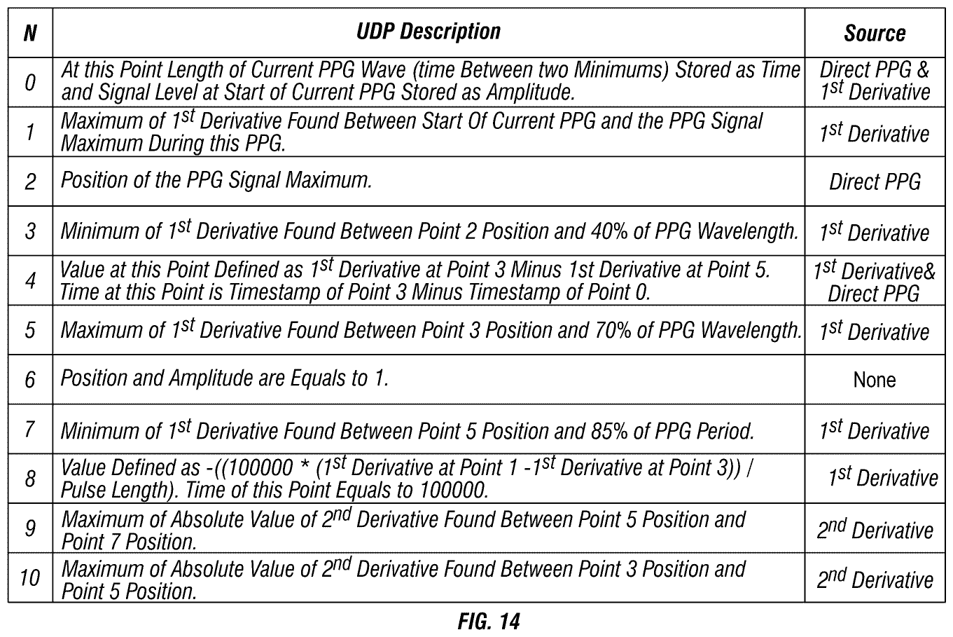

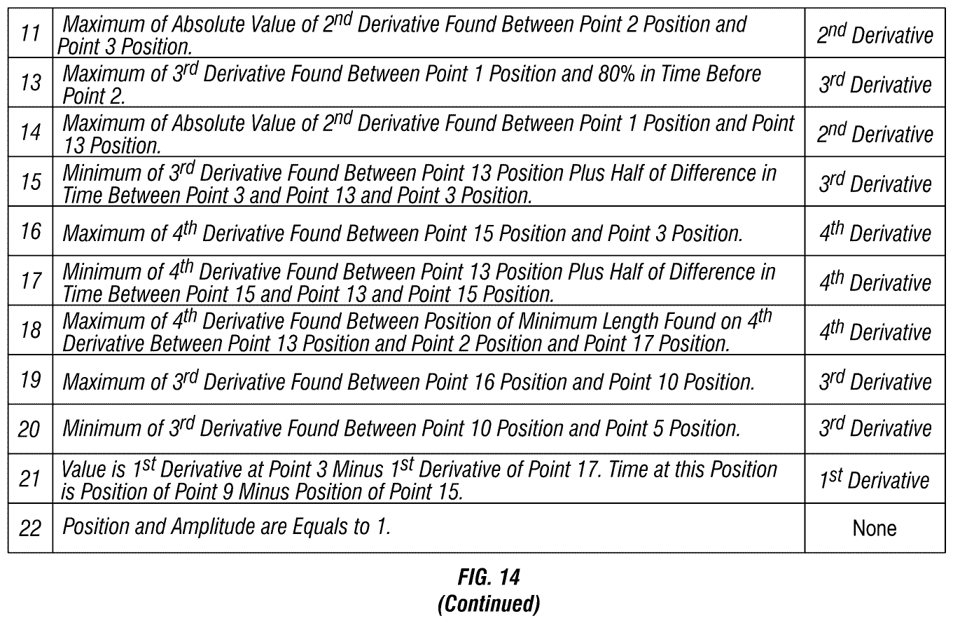

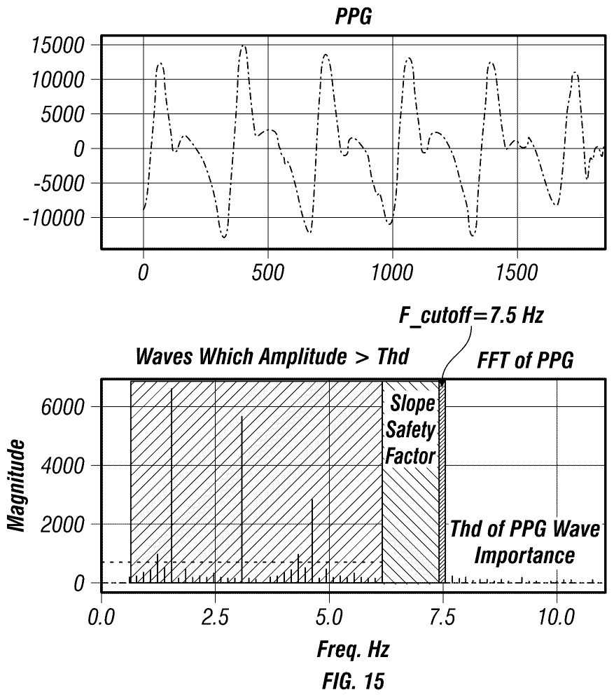

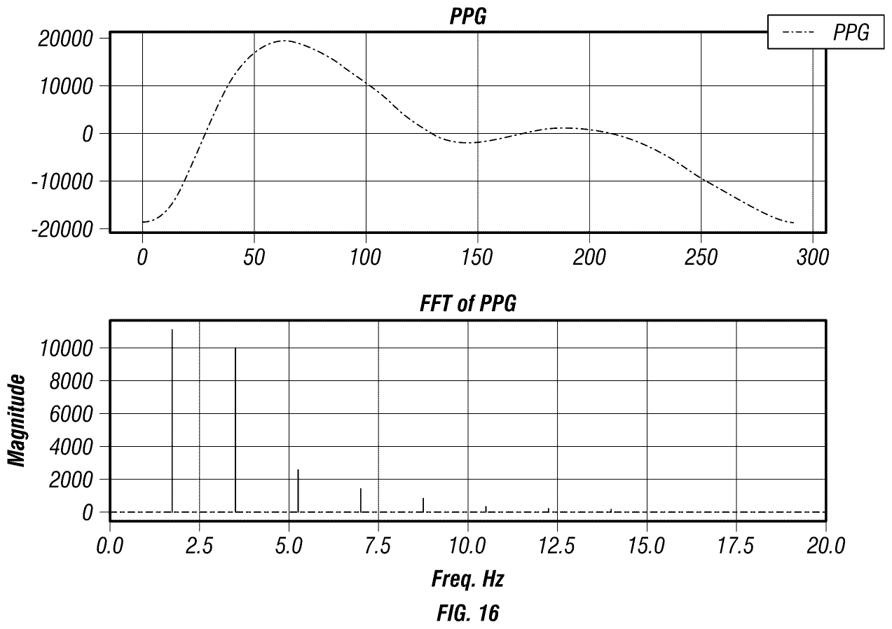

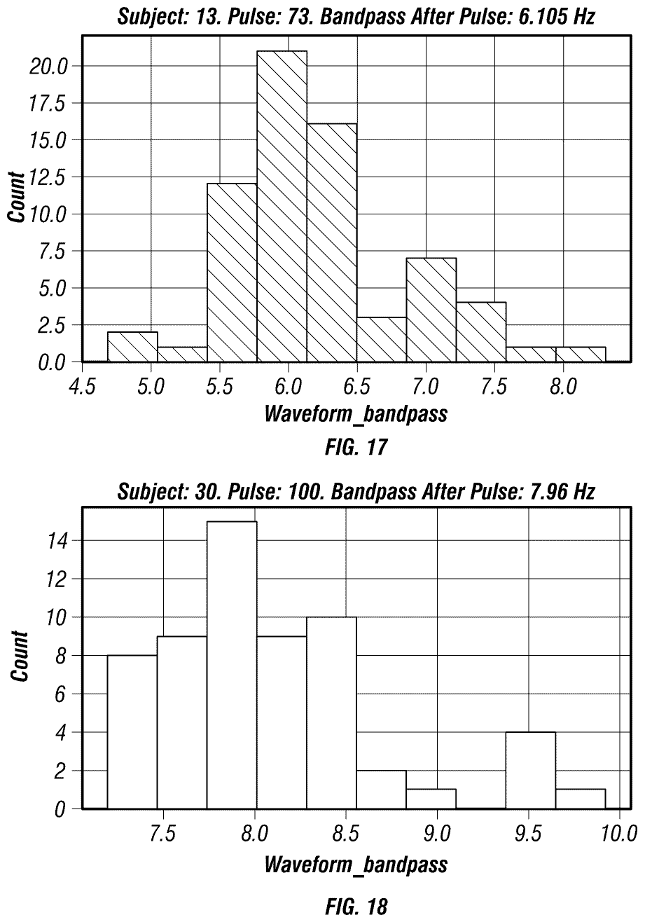

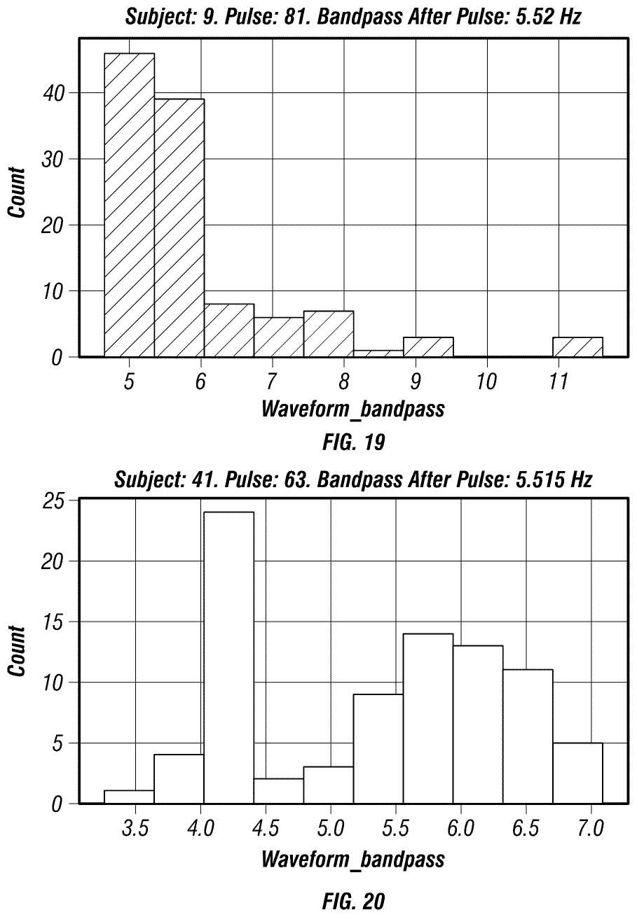

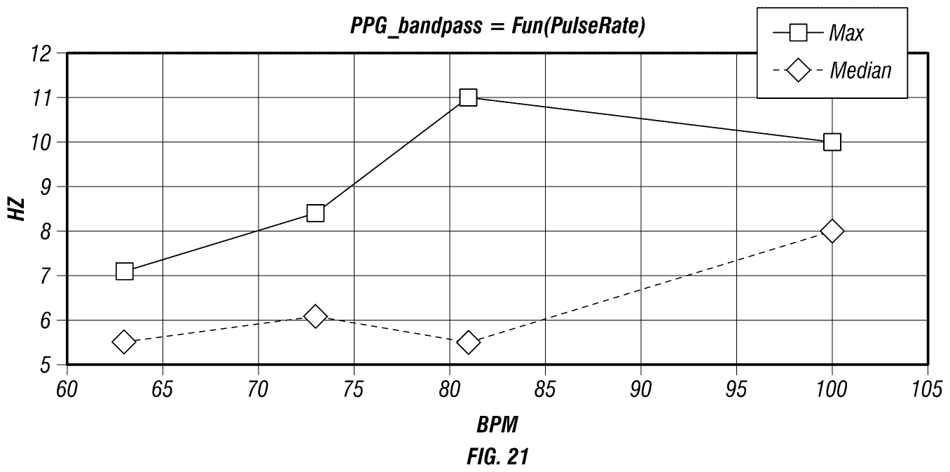

FIG. 15 shows the PPG coming from the optical sensor and undergoing a Fast Fourier Transfer (FFT) spectral analysis. The lower figure shows different frequencies (x-axis) and their intensities (y-axis). The red dotted line is the threshold under which all signals are ignored due to their low intensities, and considered a noise, while above it all are strong enough signals to be taken into account as useful. Such a threshold (dotted line) is identified by the clinical researcher based on the statistical picture of the subject/patient. The cut-off frequency is identified as a frequency above which all signals are considered a noise and are getting filtered. FIG. 16 is a demonstration of FFT analysis on one PPG sample with the identified by the spectral analysis frequencies (the lower plot). FIGS. 17 and 18 show the histogram of distribution of each identified in the PPG signal frequencies around a main applied cut-off frequency of 6.105 Hz (FIG. 17), and after applying a cut-off frequency 7.96 Hz (FIG. 18). These frequencies have been identified on specific two different subjects respectively, as an example of spectral analysis with FFT, with cut-off frequency identification. FIGS. 19 and 20 show the results after signal filtering on two other subjects. Additionally, a relationship between the heart rate (Pulse Rate) and the cut-off frequency is established (FIG. 21). Such relationship is correlating with the FFT-based cut-off frequency identification described herein.

The method 1200 then advances to block 1224 in which an outlier filter could be applied. For example, in some embodiments, every new input parameter value could be added to FIFO. A median and median absolute deviation (MAD) may be calculated for FIFO. In some cases, this could be adding the new value to the moving window average if abs (value−median)<(3*1.4826*MAD), otherwise FIFO median is added instead of the new value to the moving window average.

The method 1200 may then advance to block 1226 in which a moving window average could be applied. For example, there could be moving window averaging over 32 conditioned heartbeats/PPG signals. In some cases, every new value is added to the FIFO N values size. The arithmetic mean of the values in the FIFO is calculated. If the FIFO is more than 33% full, the arithmetic mean is the output value. Otherwise, no value is returned.

Some non-limiting examples of the above-described embodiments can include the following:
1. A wearable device comprising:
a plurality of photoplethysmogram (“PPG”) channels, wherein each of the plurality of PPG channels comprise at least one light emitter and at least one light receiver sensor;
a non-transitory computer-readable memory having computer instructions stored thereon;
a processor in communication with the plurality of PPG channels and the computer-readable memory, wherein the computer instructions, when executed by the processor, causes the processor to perform operations comprising:
   generating respective PPG signals of the plurality of PPG channels;
   establishing one or more selection parameters to choose a selected PPG channel;
   choosing the selected PPG channel from the plurality of PPG channels based on which of the respective PPG signals best fits the one or more selection parameters; and
   transmitting data associated with the PPG signal of the selected PPG channel.
2. The wearable device of 1, wherein the one or more selection parameters includes a noise to signal ratio of a PPG signal generated by the selected PPG channel.
3. The wearable device of 1, wherein the one or more selection parameters includes a power consumption of a PPG signal generated by the selected PPG channel.
4. The wearable device of 1, wherein the one or more selection parameters includes an amplitude of a PPG signal generated by the selected PPG channel.
5. The wearable device of 1, wherein the at least one light emitter comprises one or more of an infrared LED and/or a visible light LED.
6. The wearable device of 1, wherein the wearable device is a ring.
7. The wearable device of 6, wherein the at least one light emitter of the plurality of PPG channels are spatially arranged along the ring to emit light towards a finger of a user.
8. A photoplethysmogram (“PPG”) ring configured to be worn on a finger of a user, the PPG ring comprising:
a ring body including an inner body and an outer body;
a flexible circuit disposed between the inner body and the outer body of the ring body, wherein the flexible circuit comprises a plurality of light emitters to selectively transmit light and a plurality of light receiver sensors arranged along the inner surface to detect the transmitted light, wherein the plurality of light emitters and a plurality of light receiver sensors define a plurality of PPG channels;
a non-transitory computer-readable memory having computer instructions stored thereon;
a processor in communication with the plurality of PPG channels and the computer-readable memory, wherein the computer instructions, when executed by the processor, causes the processor to perform operations comprising:
   generating respective PPG signals of the plurality of PPG channels;
   establishing one or more selection parameters to choose a selected PPG channel;
   choosing a selected PPG channel from the plurality of PPG channels based on which of the respective PPG signals best fits the one or more selection parameters; and
   transmitting data associated with the PPG signal of the selected PPG channel.
9. The PPG ring of 8, further comprising computer instructions to prioritize the plurality of PPG channels based on power consumption.
10. The PPG ring of 9, further comprising computer instructions to choose the selected PPG channel based on the noise to signal ratio in the respective PPG signals as a function of power consumption prioritization.
11. The PPG ring of 9, further comprising computer instructions to choose the selected PPG channel based on an amplitude in the respective PPG signals as a function of power consumption prioritization.
12. The PPG ring of 9, wherein at least a portion of the plurality of light emitters comprises one or more of an infrared LED and/or a visible light LED, and the plurality of light emitters are configured to transmit infrared and/or visible light through one or more openings defined in the inner body.
13. The PPG ring of 9, wherein at least a portion of the PPG channels includes illumination of at least one infrared LED and at least one visible light LED.
14. The PPG ring of 13, where at least a portion of the PPG channels includes illumination of at least two infrared LEDs and at least two visible light LEDs.
15. The PPG ring of 14, wherein at least a portion of the light receiver sensors are arranged on approximately opposing sides of the inner body.
16. A method of selecting a photoplethysmogram (“PPG”) channel, the method comprising:
receiving respective PPG signals of a plurality of PPG channels, wherein the PPG signals were generated based on one or more light emitters and one or more light receiver sensors;
establishing one or more selection parameters to choose a selected PPG channel;
choosing the selected PPG channel from the plurality of PPG channels based on which of the respective PPG signals best fits the one or more selection parameters; and
transmitting data associated with the PPG signal of the selected PPG channel.
17. The method of 16, wherein the one or more selection parameters includes a noise to signal ratio of a PPG signal generated by the selected PPG channel.
18. The method of 16, wherein the one or more selection parameters includes a power consumption of a PPG signal generated by the selected PPG channel.
19. The method of 16, wherein the one or more light emitters comprises one or more of an infrared LED and/or a visible light LED.
20 The method of 16, wherein the plurality of PPG channels comprises at least one PPG channel with at least two infrared LEDs and at least one PPG channel with at least two visible light LEDs.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

1. A wearable device comprising:
a plurality of photoplethysmogram (“PPG”) channels, wherein each of the plurality of PPG channels comprise at least one light emitter and at least one light receiver sensor;
a non-transitory computer-readable memory having computer instructions stored thereon;
a processor in communication with the plurality of PPG channels and the computer-readable memory, wherein the computer instructions, when executed by the processor, causes the processor to perform operations comprising:
generating respective PPG signals of the plurality of PPG channels;
establishing one or more selection parameters to choose a selected PPG channel;
choosing the selected PPG channel from the plurality of PPG channels based on which of the respective PPG signals best fits the one or more selection parameters; and
transmitting data associated with the PPG signal of the selected PPG channel.

2. The wearable device of claim 1, wherein the one or more selection parameters includes a noise to signal ratio of a PPG signal generated by the selected PPG channel.

3. The wearable device of claim 1, wherein the one or more selection parameters includes a power consumption of a PPG signal generated by the selected PPG channel.

4. The wearable device of claim 1, wherein the one or more selection parameters includes an amplitude of a PPG signal generated by the selected PPG channel.

5. The wearable device of claim 1, wherein the at least one light emitter comprises one or more of an infrared LED and/or a visible light LED.

6. The wearable device of claim 1, wherein the wearable device is a ring.

7. The wearable device of claim 6, wherein the at least one light emitter of the plurality of PPG channels are spatially arranged along the ring to emit light towards a finger of a user.

8. A photoplethysmogram (“PPG”) ring configured to be worn on a finger of a user, the PPG ring comprising:
a ring body including an inner body and an outer body;
a flexible circuit disposed between the inner body and the outer body of the ring body, wherein the flexible circuit comprises a plurality of light emitters to selectively transmit light and a plurality of light receiver sensors arranged along the inner surface to detect the transmitted light, wherein the plurality of light emitters and a plurality of light receiver sensors define a plurality of PPG channels;
a non-transitory computer-readable memory having computer instructions stored thereon;
a processor in communication with the plurality of PPG channels and the computer-readable memory, wherein the computer instructions, when executed by the processor, causes the processor to perform operations comprising:
generating respective PPG signals of the plurality of PPG channels;
establishing one or more selection parameters to choose a selected PPG channel;
choosing a selected PPG channel from the plurality of PPG channels based on which of the respective PPG signals best fits the one or more selection parameters; and
transmitting data associated with the PPG signal of the selected PPG channel.

9. The PPG ring of claim 8, further comprising computer instructions to prioritize the plurality of PPG channels based on power consumption.

10. The PPG ring of claim 9, further comprising computer instructions to choose the selected PPG channel based on the noise to signal ratio in the respective PPG signals as a function of power consumption prioritization.

11. The PPG ring of claim 9, further comprising computer instructions to choose the selected PPG channel based on an amplitude in the respective PPG signals as a function of power consumption prioritization.

12. The PPG ring of claim 9, wherein at least a portion of the plurality of light emitters comprises one or more of an infrared LED and/or a visible light LED, and the plurality of light emitters are configured to transmit infrared and/or visible light through one or more openings defined in the inner body.

13. The PPG ring of claim 9, wherein at least a portion of the PPG channels includes illumination of at least one infrared LED and at least one visible light LED.

14. The PPG ring of claim 13, where at least a portion of the PPG channels includes illumination of at least two infrared LEDs and at least two visible light LEDs.

15. The PPG ring of claim 14, wherein at least a portion of the light receiver sensors are arranged on approximately opposing sides of the inner body.

16. A method of selecting a photoplethysmogram (“PPG”) channel, the method comprising:
receiving respective PPG signals of a plurality of PPG channels, wherein the PPG signals were generated based on one or more pairs of light emitters and light receiver sensors;
establishing one or more selection parameters to choose a selected PPG channel;
choosing the selected PPG channel from the plurality of PPG channels based on which of the respective PPG signals best fits the one or more selection parameters; and
transmitting data associated with the PPG signal of the selected PPG channel.

17. The method of claim 16, wherein the one or more selection parameters includes a noise to signal ratio of a PPG signal generated by the selected PPG channel.

18. The method of claim 16, wherein the one or more selection parameters includes a power consumption of a PPG signal generated by the selected PPG channel.

19. The method of claim 16, wherein the one or more light emitters comprises one or more of an infrared LED and/or a visible light LED.

20. The method of claim 16, wherein the plurality of PPG channels comprises at least one PPG channel with at least two infrared LEDs and at least one PPG channel with at least two visible light LEDs.
